(19) United States
(12) Patent Application Publication
(10) Pub. No.: US 2021/0346246 A1
(43) Pub. Date: Nov. 11, 2021

(54) SOLID COSMETIC COMPOSITION COMPRISING ISOMALT AND THICKENING AGENT

(71) Applicant: COSMETIC WARRIORS LIMITED, Poole, Dorset (GB)

(72) Inventors: Mark CONSTANTINE, Poole, Dorset (GB); Margaret Joan CONSTANTINE, Poole, Dorset (GB); Helen Elizabeth AMBROSEN, Poole, Dorset (GB); Rowena Jacqueline BIRD, Poole, Dorset (GB); Alessandro COMMISSO, Poole, Dorset (GB)

(21) Appl. No.: 17/278,159

(22) PCT Filed: Sep. 20, 2019
(86) PCT No.: PCT/GB2019/052646

§ 371 Date: Mar. 19, 2021

(30) Foreign Application Priority Data
Sep. 21, 2018 (GB) 1815420.3

(51) Int. Cl. A61K 8/02; A61K 8/34; A61K 8/73; A61K 8/81; A61K 8/41; A61Q 11/00; A61Q 5/06

(52) U.S. Cl. CPC A61K 8/0216; A61K 8/345; A61K 8/733; A61K 8/737; A61K 8/8176; A61K 2800/92; A61Q 11/00; A61Q 5/06; A61K 2800/48; A61K 2800/591; A61K 2800/805; A61K 8/416

(57) ABSTRACT

A solid cosmetic composition includes (i) isomalt in an amount of from about 40% to about 90% by weight of the solid cosmetic composition; and (ii) a thickening agent in an amount of from about 1% to about 30% by weight of the solid cosmetic composition. The thickening agent is selected from the group consisting of a hydrocolloid, a clay, and mixtures thereof.

### FIELD OF THE INVENTION

The present invention relates to a solid cosmetic composition, a process for producing said solid cosmetic composition, a product prepared by the process, and a cosmetic method for using the solid cosmetic composition. The present invention also relates to a use of isomalt.

### BACKGROUND TO THE INVENTION

The present invention relates to solid cosmetic compositions, particularly those for use in contact with the human or animal body.

In the modern era, a high proportion of cosmetic products are typically provided to consumers in the form of liquid or pastes. For example, hair styling products are typically provided as hair gels or pastes, which require packaging in order to transport and store said products. Oral care (or oral hygiene) products, such as toothpastes and mouthwashes, are also either provided in paste or liquid form, thus requiring some form of external packaging. They are sold in containers to the end user and may be dispensed by the end user. However, the required use of packaging is a disadvantage. From an environmental perspective, waste packaging is a significant problem, despite the availability of recycling.

There is also a desire to provide more ‘exciting’ products which add to the bathing, cleansing and/or grooming experience of a user. In other words, there is a desire to provide products which offer to the user more than the functional delivery of a cosmetically active agent.

The present invention seeks to provide a solid cosmetic product, which avoids the need to use external packaging, whilst providing the user with a more exciting delivery of the cosmetic material.

### SUMMARY OF THE INVENTION

According to a first aspect of the invention, there is provided a solid cosmetic composition comprising (i) isomalt in an amount of from about 40% to about 90% by weight of the solid cosmetic composition; and (ii) a thickening agent in an amount of from about 1% to about 30% by weight of the solid cosmetic composition, and wherein the thickening agent is selected from the group consisting of a hydrocolloid, a clay, and mixtures thereof.

According to a second aspect of the invention, there is provided a process for the production of a solid cosmetic composition according to the first aspect of the invention, the process comprising the steps of:
(a) combining:
   (i) isomalt, and
   (ii) a thickening agent; and
(b) solidifying the resultant composition.

According to a third aspect of the invention, there is provided a solid cosmetic composition obtained or obtainable by a process, the process comprising the steps of:
(a) combining:
   (i) isomalt, and
   (ii) a thickening agent; and
(b) solidifying the resultant composition.

According to a fourth aspect of the invention, there is provided a cosmetic method comprising contacting the skin, hair or teeth of a user with the solid cosmetic composition according to the first aspect of the invention.

The present inventors have found that the combination of isomalt with a thickening agent provides a solid cosmetic composition that has acceptable solidity and hardness that it may be handled and transported easily, without requiring the use of any external packaging. It was also found that the solid product may have a desirable feel for the user, thus providing a pleasant experience during use. In particular, it was found that the inclusion of isomalt in the product provided a composition having sufficient solidity, whilst also having an enhanced shelf-life. Without wishing to be bound, this may be due in part to the non-hygroscopic nature of isomalt, which inhibits absorption of water from surrounding moisture in the air by the product during storage at room temperature. The product may thus be more resistant to microbiological growth without requiring the use of preservatives. It was also found that the solid cosmetic composition does not become sticky or tacky on the outside after an extended period of time without being contained in any external packaging during storage. This means that the product does not require external packaging, even when left on a shelf for several weeks.

It was also surprisingly found that the specific combination of isomalt with a thickening agent provides a solid composition that releases cosmetic material that the user may apply to their body (e.g. their skin, hair and/or teeth) upon contact of the solid cosmetic composition with water. Upon contact with water, cosmetic material (e.g. a cosmetically active agent, such as a surfactant, oral care active, hair gelling active, or the like) may be released at the surface on the product in the form of a non-solid (e.g. semi-solid or liquid) material. For example, the cosmetic material may be released at the surface in the form of a gel or paste. This may then be applied by the user to their body (e.g. skin, hair and/or teeth) as desired.

It is particularly noted that, even after contacting the solid composition with water, the composition is able to dry after a relatively short period of time (from about 1 hour to about 24 hours), so as to provide a non-tacky and dry solid composition. For example, where the cosmetic material at the surface of the solid composition has been converted from the solid phase to a semi-solid or liquid phase, this semi-solid or liquid phase may subsequently dry when left at room temperature and ambient humidity so as to re-solidify (i.e. the whole composition becomes solid again, including the material at the surface). In this way, the composition may be reused.

For ease of reference, these and further aspects of the present invention are now discussed under appropriate section headings. However, the teachings under each section are not necessarily limited to each particular section.

### DETAILED DESCRIPTION

Composition

As described herein, in one aspect of the present invention, there is provided a solid cosmetic composition comprising: (i) isomalt in an amount of from about 40% to about 90% by weight of the solid cosmetic composition; and (ii) a thickening agent in an amount of from about 1% to about 30% by weight of the solid cosmetic composition, and wherein the thickening agent is selected from the group consisting of a hydrocolloid, a clay, and mixtures thereof.

It will be understood by one skilled in the art that the nature of a cosmetic product means that the product is not edible. Thus, the present invention provides a non-edible solid cosmetic composition as defined herein.

Solid cosmetic products of the present invention are compositions which can substantially sustain their physical shape when unsupported by external means, e.g. packaging etc. Thus, they are considered to be solid, solid-like, in solid form or in solid-like form at room temperature. For the avoidance of doubt, the solid product is one which remains substantially solid at a temperature of up to 30° C.

By solid-like, it is understood that some materials are considered on a day to day basis to be solid, yet over an extremely long period of time, may alter in shape, e.g. amorphous materials such as glass etc. However, they are considered to be solid-like as, for the purpose they fulfill, they are solid.

As mentioned above, due to the solid form of the compositions of the present invention, external packaging is not required to maintain the shape of the composition.

Isomalt

As described herein, the solid cosmetic composition comprises isomalt. Isomalt is a polyol, commonly referred to in the art as a sugar alcohol. The IUPAC name for isomalt is (2R,3R,4R,5R)-6-[[(2S,3R,4S,5S,6R)-3,4,5-trihydroxy-6-(hydroxymethyl)-2-tetrahydropyranyl]oxy]hexane-1,2,3,4,5-pentol. Isomalt is a derivative of sucrose, and is typically an equimolar mixture of two mutually diastereomeric disaccharides, each composed of two sugars: glucose and mannitol (α-D-glycopyranosyl-1,1-D-mannitol (GPM)), and glucose and sorbitol (α-D-glucopyranosyl-1,6-D-sorbitol (GPS)).

Isomalt is typically manufactured in a two-stage process in which sucrose is first transformed by enzymatic transglucosidation into isomaltulose, a reducing disaccharide (6-O-α-D-glucopyranosyl-D-fructose). The isomaltulose is then hydrogenated into isomalt using a Raney nickel catalyst.

As the skilled person will appreciate, isomalt is a sugar alcohol that has a very low hygroscopicity. It is commonly regarded as being “non-hygroscopic”, particularly at ambient temperatures and humidities, This means that isomalt will not absorb water from its surroundings very readily. Indeed, at 20° C., isomalt will not absorb a significant amount of water from its surroundings (i.e. its water content will be very low) until a relative humidity of at least about 85% is reached. Even at 25° C., isomalt absorbs virtually no water up to a relative humidity of 85%. Isomalt does not start absorbing water until the temperature reaches from about 60° C. to about 80° C. at about 75% to about 65% relative humidity.

Isomalt typically has a high melting temperature range of from about 145° C. to about 150° C., and thus is considered to be relatively resistant to heat.

As described herein, the solid cosmetic composition comprises isomalt in an amount of from about 40% to about 90% by weight of the solid cosmetic composition. In some embodiments, the solid cosmetic composition comprises isomalt in an amount of from about 45% to about 90% by weight of the solid cosmetic composition. In some embodiments, the solid cosmetic composition comprises isomalt in an amount of from about 45% to about 85% by weight of the solid cosmetic composition. In some embodiments, the solid cosmetic composition comprises isomalt in an amount of from about 45% to about 80% by weight of the solid cosmetic composition. In some embodiments, the solid cosmetic composition comprises isomalt in an amount of from about 45% to about 75% by weight of the solid cosmetic composition. In some embodiments, the solid cosmetic composition comprises isomalt in an amount of from about 50% to about 70% by weight of the solid cosmetic composition. In some embodiments, the solid cosmetic composition comprises isomalt in an amount of from about 50% to about 65% by weight of the solid cosmetic composition. In some embodiments, the solid cosmetic composition comprises isomalt in an amount of from about 55% to about 80% by weight of the solid cosmetic composition. In some embodiments, the solid cosmetic composition comprises isomalt in an amount of from about 55% to about 75% by weight of the solid cosmetic composition. In some embodiments, the solid cosmetic composition comprises isomalt in an amount of from about 60% to about 75% by weight of the solid cosmetic composition.

It has been surprisingly found by the present inventors that the inclusion of isomalt provides a sufficiently solid product having an acceptable shelf-life, whilst also enabling sufficient release of the cosmetic material from the solid composition upon contacting the composition with water. Indeed, it was found by the present inventors that other sugar alcohols (such as sorbitol and xylitol) were not able to provide a solid product that did not require any external packaging. Sugar itself was not found to be suitable as the product became increasingly tacky and sticky over time, eventually deteriorating to a paste when left outside of packaging during storage. Furthermore, less hygroscopic sugar alcohols (such as mannitol) were found not to be suitable in the present instance as it was not found to be possible to release a sufficient amount of the cosmetic material (e.g. active agent) in the form of a gel or paste upon contact with water.

For the avoidance of doubt, as used herein, “release of cosmetic material” means that the cosmetic components in the solid composition may be transformed from a solid phase into a semi-solid (e.g. gel/paste) or liquid phase at the surface of the solid composition. This semi-solid or liquid phase may then be capable of being applied (e.g. by spreading or rubbing) to the body of the user.

Thickening Agent

As described herein, the solid cosmetic composition comprises a thickening agent.

As described herein, the thickening agent is selected from the group consisting of a hydrocolloid, a clay, and mixtures thereof. In some embodiments, the thickening agent is a hydrocolloid or a clay.

The thickening agent may be a hydrocolloid. As the skilled person will appreciate, hydrocolloids are a heterogeneous group of long chain polymers characterised by their property of forming viscous dispersions and/or gels when dispersed in water. Some hydrocolloids have the ability to modify the rheology of compositions comprising water. There are two types of hydrocolloid: gelling agents (i.e. those that modify the texture and/or solidity of the composition) and thickening agents (i.e. those that modify the viscosity of the composition). The water-thickening property is common to all hydrocolloids, and thus gelling agents can be regarded as a subset of thickening agents. In some embodiments, the thickening agent is also a gelling agent, such as carrageenan, agar, pectin, a combination of a salt of alginic acid and calcium ions, calcium alginate, gellan gum, methyl cellulose, hydroxypropyl methylcellulose, modified starch, and mixtures thereof. In some embodiments, the thickening agent is a hydrocolloid that modifies the viscosity of the composition but which is not a gelling agent, such as xanthan gum, guar gum, konjac gum, gum tragacanth and gum Arabic.

In some embodiments, the thickening agent is a polysaccharide or mixture of polysaccharides.

In some embodiments, the thickening agent is a clay, preferably a bentonite and/or hectorite clay.

In some embodiments, the thickening agent is selected from the group consisting of xanthan gum, cationic guar gum, agar, pectin, a salt of alginic acid, carrageenan (including κ-carrageenan, ι-carrageenan and λ-carrageenan), Arabic gum, acacia gum, locust bean gum, tars gum, konjac, tragacanth gum, karaya gum, gellan gum, cellulose, derivatives of cellulose (including carboxymethylcellulose, methylcellulose, hydroxypropylcellulose, methylhydroxypropylcellulose, microcrystalline cellulose), bentone, hectorite clay, and mixtures thereof. The thickening agent may preferably be selected from the group consisting of xanthan gum, cationic guar gum, agar, pectin, a salt of alginic acid, carrageenan, Arabic gum, acacia gum and mixtures thereof. The salt of alginic acid may be a salt of a monovalent cation; for example, sodium alginate or potassium alginate. The salt of alginic acid may alternatively or additionally be calcium alginate, ammonium alginate or propylene glycol alginate.

In some embodiments, the thickening agent is selected from the group consisting of xanthan gum, cationic guar gum, carrageenan, a salt of alginic acid, and mixtures thereof. In some embodiments, the thickening agent is selected from the group consisting of xanthan gum, cationic guar gum, carrageenan, sodium alginate, and mixtures thereof. In some embodiments, the thickening agent is selected from the group consisting of xanthan gum, cationic guar gum, carrageenan, and mixtures thereof. In some embodiments, the thickening agent is cationic guar gum. In some embodiments, the thickening agent is xanthan gum. In some embodiments, the thickening agent is sodium alginate.

As described herein, the thickening agent (such as xanthan gum) is present in an amount of from about 1% to about 30% by weight of the solid cosmetic composition. In some embodiments, the thickening agent (such as xanthan gum) is present in an amount of from about 1% to about 25% by weight of the solid cosmetic composition. In some embodiments, the thickening agent (such as xanthan gum) is present in an amount of from about 1% to about 20% by weight of the solid cosmetic composition. In some embodiments, the thickening agent (such as xanthan gum) is present in an amount of from about 2% to about 15% by weight of the solid cosmetic composition. In some embodiments, the thickening agent (such as xanthan gum) is present in an amount of from about 2.5% to about 10% by weight of the solid cosmetic composition. In some embodiments, the thickening agent (such as xanthan gum) is present in an amount of from about 2.5% to about 5% by weight of the solid cosmetic composition.

It was found by the present inventors that, when a thickening agent is combined with isomalt, a solid cosmetic composition may be produced which releases some cosmetic material upon contact of the composition with water. For example, the cosmetic material may be released at the surface of the composition in the form of a semi-solid or liquid (e.g. gel or paste) that is spreadable on the body of the user.

As described herein, the thickening agent (such as xanthan gum) is present in an amount of from about 1% to about 30% by weight of the solid cosmetic composition, and more preferably in an amount of from about 1% to about 20% by weight of the solid cosmetic composition. It was found that, when the thickening agent was included in an amount of at least 1% by weight of the composition, a solid product may be formed that provides an acceptable release of the cosmetic material upon contact with water. When the thickening agent was included in an amount of no greater than 20% by weight, it was also found that the solidity of the product was improved.

Humectant

In some preferred embodiments, the solid cosmetic composition further comprises a humectant. It was found by the present inventors that the inclusion of a humectant not only improves the moisturising properties of the product, but may also improve the ease of release of the cosmetic material from the solid composition upon contact with water.

In some embodiments, the solid cosmetic composition further comprises a humectant selected from the group consisting of glycerine, propylene glycol, 1,3-propanediol, honey, sorbitol, glucose syrup, polysaccharide syrup, monosaccharide syrup, and mixtures thereof. In some embodiments, the humectant is selected from the group consisting of glycerine, propylene glycol, 1,3-propanediol, honey, sorbitol, and mixtures thereof. In some embodiments, the humectant is selected from the group consisting of glycerine, propylene glycol, and mixtures thereof.

When present, the humectant may be included in an amount of from about 5% to about 40% by weight of the solid cosmetic composition, such as from about 10% to about 35% by weight of the solid cosmetic composition, such as from about 15% to about 30% by weight of the solid cosmetic composition, such as from about 15% to about 25% by weight of the solid cosmetic composition, such as from about 15% to about 20% by weight of the solid cosmetic composition. When present, the humectant may be included in an amount of from about 5% to about 30% by weight of the solid cosmetic composition, such as from about 6% to about 25% by weight of the solid cosmetic composition, such as from about 7% to about 20% by weight of the solid cosmetic composition, such as from about 8% to about 20% by weight of the solid cosmetic composition.

In some preferred embodiments, the solid cosmetic composition comprises isomalt and a humectant (such as glycerine and/or propylene glycol) in a weight ratio of from about 1:1 to about 10:1. In some preferred embodiments, the solid cosmetic composition comprises isomalt and a humectant in a weight ratio of from about 1.5:1 to about 5:1. In some preferred embodiments, the solid cosmetic composition comprises isomalt and a humectant in a weight ratio of from about 2:1 to about 4:1. In some preferred embodiments, the solid cosmetic composition comprises isomalt and a humectant (such as glycerine and/or propylene glycol) in a weight ratio of from about 2:1 to about 10:1. In some preferred embodiments, the solid cosmetic composition comprises isomalt and a humectant (such as glycerine and/or propylene glycol) in a weight ratio of from about 3:1 to about 9:1

It was found in particular by the present inventors that, when the weight ratio of isomalt to humectant is from about 1:1 to about 10:1 (and in particular from about 1:1 to about 5:1), a solid cosmetic composition may be provided having an improved release of the cosmetic material from the solid composition upon contact with water, whilst providing a solid composition having a sufficient solidity (e.g. hardness).

In some embodiments, the solid cosmetic composition comprises a thickening agent (such as xanthan gum) and a humectant (such as glycerine and/or propylene glycol) in a weight ratio of from about 1:1 to about 1:15, such as from about 1:1 to about 1:10, such as from about 1:2 to about 1:8, such as from about 1:2 to about 1:6.

Active Agent

In addition to the above, the solid cosmetic composition may further comprise an active agent. As used herein, the term “active agent” means a cosmetically active agent. The active agent may be an agent that renders the composition suitable for use in cleaning the hair and/or body of a user, for use as an oral care product, for use as a hair gelling product, for use as a moisturiser or hand/body lotion, for use as a hair dye (or hair colourant), for use as a colour cosmetic (e.g. make-up, such as foundation, eye shadow, lipstick, blush or the like), or the like.

Surfactant

In some embodiments, the solid cosmetic composition further comprises a surfactant as an active agent. Products comprising a surfactant may be suitable for use in cleaning the skin, hair, and/or teeth of a user, As the skilled person will appreciate, surfactants are typically sold as commercial products in which the pure surfactant (i.e. the active surfactant) is either diluted in water or is provided as a solid product (e.g. as solid surfactant needles). As used herein, the amounts of the surfactant do not relate to the commercially available diluted products, but rather to the amount of pure surfactant.

In some embodiments, the surfactant is selected from the group consisting of anionic surfactant, amphoteric surfactant, cationic surfactant, non-ionic surfactant and mixtures thereof.

In some embodiments, the surfactant comprises an anionic surfactant. In some embodiments, the surfactant comprises an anionic surfactant selected from the group consisting of sodium lauryl sulfate, sodium laureth sulfate, sodium dodecyl sulfate, sodium cocosulfate, ammonium lauryl sulfate, ammonium laureth sulfate, sodium myreth sulfate, sodium lauroyl sarcosinate, disodium laureth sulfosuccinate, disodium lauryl sulfosuccinate, sodium lauryl sulfoacetate, sodium cocoyl isethionate, sodium olefin sulfonate, and mixtures thereof. In some embodiments, the surfactant comprises an anionic surfactant selected from the group consisting of sodium lauryl sulfate, sodium laureth sulfate, sodium cocosulfate, disodium lauryl sulfosuccinate, and mixtures thereof.

In some embodiments, the surfactant is or comprises sodium lauryl sulfate. In some embodiments, the surfactant is or comprises sodium laureth sulfate. In some embodiments, the surfactant is or comprises sodium cocosulfate, In some embodiments, the surfactant is or comprises disodium lauryl sulfosuccinate.

In some embodiments, the surfactant comprises an amphoteric surfactant. In some embodiments, the surfactant comprises an amphoteric surfactant selected from the group consisting of lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, sodium lauroyl sarcosine, cetrimonium chloride and mixtures thereof. In some embodiments, the surfactant comprises an amphoteric surfactant selected from the group consisting of lauryl betaine, cocamidopropyl betaine, sodium cocoamphoacetate, sodium lauroyl sarcosine, cetrimonium chloride and mixtures thereof. In some embodiments, the surfactant is or comprises sodium lauroyl sarcosine. In some embodiments, the surfactant is or comprises cetrimonium chloride.

In some embodiments, the surfactant is a surfactant selected from the group consisting of sodium lauryl sulfate, sodium laureth sulfate, sodium dodecyl sulfate, sodium cocosulfate, ammonium lauryl sulfate, ammonium laureth sulfate, sodium myreth sulfate, sodium lauroyl sarcosinate, disodium laureth sulfosuccinate, disodium lauryl sulfosuccinate, sodium lauryl sulfoacetate, sodium cocoyl isethionate, sodium olefin sulfonate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, sodium lauroyl sarcosine, cetrimonium chloride and mixtures thereof. In some embodiments, the surfactant is a surfactant selected from the group consisting of sodium lauryl sulfate, sodium laureth sulfate, sodium cocosulfate, disodium lauryl sulfosuccinate, sodium lauroyl sarcosine, cetrimonium chloride, and mixtures thereof. In some embodiments, the surfactant is sodium lauryl sulfate. In some embodiments, the surfactant is sodium laureth sulfate. In some embodiments, the surfactant is sodium cocosulfate. In some embodiments, the surfactant disodium lauryl sulfosuccinate. In some embodiments, the surfactant is sodium lauroyl sarcosine. In some embodiments, the surfactant is or comprises cetrimonium chloride.

In some embodiments, the surfactant (such as sodium lauryl sulfate, sodium laureth sulfate, sodium cocosulfate, disodium lauryl sulfosuccinate, sodium lauroyl sarcosine, cetrimonium chloride, and mixtures thereof) is present in an amount of from about 0.1% to about 40% by weight of the solid cosmetic composition, such as from about 0.5% to about 35% by weight of the solid cosmetic composition, such as from about 1% to about 30% by weight of the solid cosmetic composition, such as from about 5% to about 25% by weight of the solid cosmetic composition, such as from about 5% to about 20% by weight of the solid cosmetic composition, such as from about 7.5% to about 15% by weight of the solid cosmetic composition, such as from about 7.5% to about 10% by weight of the solid cosmetic composition. Preferably, the surfactant (such as sodium lauryl sulfate, sodium laureth sulfate, sodium cocosulfate, disodium lauryl sulfosuccinate, sodium lauroyl sarcosine, cetrimonium chloride, and mixtures thereof) is present in an amount of from about 5% to about 25% by weight of the solid cosmetic composition.

The inclusion of a surfactant may render the product suitable for use in cleaning the hair, skin and/or teeth of the user, Therefore, in some embodiments, the solid cosmetic composition comprises a surfactant as an active agent, and is a solid shower gel, solid shampoo and/or solid oral care product (such as a solid toothpaste).

Hair Gelling Agent

In some embodiments, the solid cosmetic composition further comprises a hair gelling agent as an active agent. In some embodiments, the solid cosmetic composition further comprises polyvinylpyrrolidone (PVP), PVP and dimethylaminoethylmethylacrylate copolymer, carbomer, vinylpyrrolidone (VP) and vinyl acetate (VA) copolymer, VP and dimethylaminopropylamine (DMAPA) Acrylates copolymer, polyacrylate-2-crosspolymer, sodium polyitaconate, VP/methacrylamide/vinyl imidazole copolymer, PVP K-90, or mixtures thereof. Preferably the hair gelling agent is PVP.

In some embodiments, the hair gelling agent (such as PVP) is present in an amount of from about 0.1% to about 10% by weight of the solid cosmetic composition, such as in an amount of from about 1% to about 7.5% by weight of the solid cosmetic composition, such as in an amount of from about 2.5% to about 5% by weight of the solid cosmetic composition.

Oral Care Agent

In some embodiments, the solid cosmetic composition further comprises an oral care agent as an active agent. For the avoidance of doubt, as used herein “oral care” is synonymous with oral hygiene. in such embodiments, the solid cosmetic composition is a solid oral care product, such as a solid toothpaste or solid mouthwash. In such embodiments, the solid cosmetic composition is thus suitable for use in cleaning or whitening the teeth of the user and/or freshening the breath of the user.

The oral care agent may be selected from the group consisting of calcium carbonate, aluminium hydroxide, calcium hydrogen phosphate, silica, zeolite, sodium fluoride, stannous fluoride, olaflur, sodium monofluorophosphate, xylitol, sodium bicarbonate, and mixtures thereof. In some embodiments, the oral care agent is selected from the group consisting of calcium carbonate, calcium hydrogen phosphate, silica, sodium fluoride, xylitol, sodium bicarbonate, and mixtures thereof.

In some embodiments, the solid cosmetic composition comprises an oral care agent (such as calcium carbonate, calcium hydrogen phosphate, silica, sodium fluoride, xylitol, sodium bicarbonate, or mixtures thereof) in an amount of from about 0.1% to about 15% by weight of the solid cosmetic composition, such as in an amount of from about 1% to about 10% by weight of the solid cosmetic composition, such as in an amount of from about 5% to about 10% by weight of the solid cosmetic composition.

For the avoidance of any doubt, it is noted that, when the solid cosmetic composition comprises an oral care agent, the solid cosmetic composition may further comprise a surfactant (and vice versa). Therefore, in some embodiments, the solid cosmetic composition further comprises a surfactant and an oral care agent as active agents.

Hair Colouring Agent

In some embodiments, the solid cosmetic composition further comprises a hair colouring agent as an active agent. The colouring can be naturally derived or it can be synthetic. Preferably it is naturally occurring. As will be appreciated by one skilled in the art a hair colouring is commonly referred to as a ‘dye’, and is preferably any colouring that is completely or significantly soluble in an aqueous medium. The ‘Ecological and Toxicological Association of Dyes and Pigment Manufacturers’ defines a dye as an “intensely coloured or fluorescent organic substances only, which impart colour to a substrate by selective absorption of light. They are typically soluble and/or go through an application process, which, at least temporarily, destroys any crystal structure by absorption, solution, and mechanical retention, or by ionic or covalent chemical bonds”.

Natural hair colourants are known to those skilled in the art and are materials and compositions which are naturally occurring and have the ability to colour compositions. In one aspect, the natural colourant is a colourant derived from organic sources. In one aspect, the natural hair colourant is a colourant not derived from inorganic sources. In one aspect, the natural hair colourant is a colourant derived from organic, and not inorganic, sources. In one aspect, the natural hair colourant is an organic pigment. The terms organic and inorganic are understood by those skilled in the art. Typically, an organic material is considered to be a material containing carbon. An inorganic material is one that is not organic.

In one aspect, the natural hair colourant is a colourant that is derived from plant-based material. In one aspect, the natural hair colourant is a colourant that a component of a plant. In one aspect the natural colourant is selected from beetroot, chlorophyll, gardenia, blackberry, coffee, rose, caramel powder, grape, alfalfa, walnut hull, calendula, cocoa, green tea, hibiscus, kelp, olive, orange, parsley, pumpkin, spinach, spirulina, wheatgrass sources, turmeric, butterfly pea, carrot, tomato and mixtures thereof.

In one aspect the natural hair colourant is selected from chlorophyll, carotenoids, anthocyanins, betalains and mixtures thereof.

In one aspect the natural hair colourant is selected from CI Natural blue 1, CI Natural blue 2, CI Natural red 1, CI Natural red 3, CI Natural red 4, CI Natural red 6, CI Natural red 8, CI Natural red 9, CI Natural red 10, CI Natural red 11, CI Natural red 12, CI Natural red 14, CI Natural red 16, CI Natural red 18, CI Natural red 20, CI Natural red 25, CI Natural red 33, CI Natural orange 2, CI Natural orange 4, CI Natural orange 6, CI Natural yellow 2, CI Natural yellow 3, CI Natural yellow 10, CI Natural yellow 13, CI Natural yellow 16, CI Natural brown 3, CI Natural brown 7, CI Natural brown 10, CI Natural green 2, CI Natural green 3, CI Natural black 1 and mixtures thereof.

The hair colouring may be an oxidative or non-oxidative dye. Preferably, the hair colouring is a non-oxidative dye. In some embodiments, the hair colouring is a non-oxidative dye selected from Acid Yellow 23, Acid Orange 7, Acid Yellow 1, Acid Red 33, Acid Red 92, Acid Violet 43, Acid Blue 9, Acid Black 1, HC Yellow 2, HC Red 3, HC Blue 2, HC Blue 15, Basic Red 51, Basic Orange 31, Basic Brown 16, Basic Brown 17, Basic Red 76, Basic Blue 99, Basic Yellow 57, Basic Yellow 87, Basic Violet 2, 4-hydroxypropylamino-3-nitrophenol, N,N′-bis-(2-hydroxyethyl)-2-nitro-phenylenediamine, and mixtures thereof. In some embodiments, the hair colouring is selected from Acid Yellow 23, Acid Orange 7, Acid Yellow 1, Acid Red 33, Acid Red 92, Acid Violet 43, Acid Blue 9, Acid Black 1, HC Yellow 2, HC Red 3, HC Blue 2, HC Blue 15, Basic Red 51, Basic Orange 31, Basic Brown 16, Basic Brown 17, Basic Red 76, Basic Blue 99, Basic Yellow 57, Basic Yellow 87, Basic Violet 2, and mixtures thereof. In some embodiments, the hair colouring is a mixture of Basic Red 51 and Basic Blue 99. In some embodiments, the hair colouring is a mixture of Basic Blue 99, Basic Brown 16, Acid Violet 43, Basic Red 76 and Basic Yellow 57. It is noted that each of these colourings are listed using the name ascribed by the International Nomenclature of Cosmetic Ingredients (INCI).

In some embodiments, the hair colouring is a colouring that is suitable for use in food. In some embodiments, the hair colouring is a colour additive that is approved for use in food, drugs and cosmetics (FD&C colourings). In some embodiments, the hair colouring is selected from FD&C Blue No. 1, FD&C Blue No. 2, FD&C Green No. 3, FD&C Red No. 3, FD&C Red No. 40, FD&C Yellow No, 5, FD&C Yellow No. 6, FD&C Green No. 1, FD&C Green No, 2, FD&C Red No, 1, FD&C Red No. 2, FD&C Red No. 4, FD&C Violet No. 1, FD&C Orange No. 1, FD&C Orange No. 2, FD&C Yellow No. 1, FD&C Yellow No. 2, FD&C Yellow No. 3, FD&C Yellow No. 4, and mixtures thereof.

In some embodiments, the hair colouring comprises a mixture of a nonoxidative dye (preferably selected from the list provided above) with a natural colourant and/or an FD&C colouring. In some embodiments, the hair colouring is a mixture of a non-oxidative dye (preferably selected from the list provided above) and an FD&C colouring.

In some embodiments, the solid cosmetic composition comprises a hair colouring agent in an amount of from about 0.1% to about 15% by weight of the solid cosmetic composition, such as in an amount of from about 1% to about 10% by weight of the solid cosmetic composition, such as in an amount of from about 1% to about 5% by weight of the solid cosmetic composition.

For the avoidance of any doubt, it is noted that, when the solid cosmetic composition comprises a hair colouring agent, the solid cosmetic composition may further comprise a surfactant (and vice versa). Therefore, in some embodiments, the solid cosmetic composition further comprises a surfactant and a hair colouring agent as active agents.

Pigment

In some embodiments, the solid cosmetic composition further comprises a pigment or skin colouring agent as an active agent.

In some embodiments, the pigment is an organic pigment, an inorganic pigment, or mixtures thereof. In some embodiments, the pigment is a natural pigment. The natural pigment may be selected from the group consisting of beetroot, chlorophyll, gardenia, blackberry, coffee, rose, caramel powder, grape, alfalfa, walnut hull, calendula, cocoa, green tea, hibiscus, kelp, olive, orange, parsley, pumpkin, spinach, spirulina, wheatgrass sources, turmeric, butterfly pea, carrot, tomato and mixtures thereof.

In some embodiments, the pigment is an inorganic pigment selected from the group consisting of iron oxide, mica (e.g. synthetic mica), titanium dioxide, tin oxide, zinc oxide, and mixtures thereof.

In some embodiments, the solid cosmetic composition comprises a pigment in an amount of from about 0.1% to about 15% by weight of the solid cosmetic composition, such as in an amount of from about 1% to about 10% by weight of the solid cosmetic composition, such as in an amount of from about 5% to about 10% by weight of the solid cosmetic composition.

Further Components

In addition to the above, the cosmetic composition may further comprise one or more cosmetically acceptable additives. The person skilled in the art is aware of a range of cosmetically acceptable additives which are suitable for incorporation into such compositions.

In some embodiments, the one or more cosmetically acceptable additives are selected from a wax, starch, binder, filler, clay, pacifier, UV absorbing material, UV reflecting material, exfoliating material (such as salt and/or sugar), essential oil, vitamin, perfume, fragrance, colouring, vegetable butter, vegetable oil, honey, fruit, fruit juice, fruit and/or herb extract, protein, decorative item, emollient, effervescent component, and mixtures thereof. Preferably, the one or more cosmetically acceptable additives are selected from the group consisting of a binder, filler, exfoliating material, essential oil, vitamin, perfume, fragrance, colouring, decorative item, emollient, effervescent component, and mixtures thereof.

Fragrance may be added to the product to make the experience of using the present composition more pleasant. Combining essential oils such as lavender, chamomile or rose absolute into fragrances for the invention ensures the user has a pleasant experience.

In some embodiments, the cosmetic composition thus further comprises a perfume or a fragrance. The amount of fragrance is preferably from about 0.1% to about 10% by weight of the total composition, such as from about 0.1% to about 5% by weight of the total composition, such as from about 0.5% to about 5% by weight of the total composition, such as from about 1% to about 5% by weight of the total composition.

The essential oils may be selected based on the fragrance desired, skin type to be treated and other effects desired based on the well-known properties of essential oils. The addition of essential oils, when taken into the nose, is known to alter mood. For example, essential oils are known to create effects of drowsiness or stimulating the senses. Many well documented effects can be achieved by the use of essential oils.

In one embodiment, the one or more essential oils present in the product are selected from Tarragon, Lemon myrtle, Jasmin, Ylang ylang, Labdanum, Lemongrass, Rose otto, Grapefruit, Patchouli, Rosemary, Armois, Lemon, Neroli, Sweet violet, Lavender, Orange 50 fold, Vanilla, Peppermint, Benzoin, Hydrangia, Litsea Cubeba, Cardamon, Tonka, and Chamomile blue. In one embodiment, the one or more essential oils present in the product are selected from Tarragon, Lemon myrtle, Labdunum, and Lemon.

Vitamins, particularly B, C and E are very beneficial for the skin. Vitamin rich ingredients such as Wheatgerm oil can also be used to deliver vitamins on to the skin. In a one embodiment, the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the product of a material, such as a natural material, that has a high vitamin content.

In some embodiments, the solid cosmetic composition further comprises a colouring. The colouring may be any pigment and/or dye suitable for imparting colour to the composition itself. In some embodiments, the solid cosmetic composition may comprise a colouring in an amount of from about 0.001% to about 5% by weight of the total composition.

In some embodiments, the solid cosmetic composition further comprises a protein. The protein may be selected from the group consisting of tofu, banana, soya, soya lecithin, hydrolysed wheat protein, and mixtures thereof. When present, the protein may be included in the solid cosmetic composition in an amount of from about 0.01% to about 10% by weight of the solid cosmetic composition, such as in an amount of from about 0.1% to about 5% by weight of the solid cosmetic composition, such as in an amount of from about 1% to about 3% by weight of the solid cosmetic composition.

In some embodiments, the solid cosmetic composition further comprises a fruit, fruit juice, and/or fruit extract. The fruit may be any suitable fruit. Preferably, the fruit may be selected from orange, mango, papaya, strawberry, banana, kiwi, apple, cherry, pineapple, raspberry, blueberry, blackberry, peach, nectarine, and mixtures thereof.

Total Composition

In some embodiments, the weight ratio of isomalt to thickening agent is from about 10:1 to about 40:1. It was found by the present inventors that, when the isomalt and thickening agent are provided in such a weight ratio, the composition has acceptable solidity and hardness that it may be handled and transported easily and also have a good feel in the hand of the user, whilst also enabling the release of a sufficient amount of cosmetic material for use by the user. For example, when the composition further comprises an active agent, the selection of the components in this weight ratio allows for a sufficient amount of the active agent to be released from the product when the product is contacted with a small amount of water (e,g, when the user applies a small amount of water thereto). If the weight ratio of isomalt to thickening agent is too low, then the product may not be sufficiently solid, and also may become tacky or sticky over time (e.g. during storage). If the weight ratio of isomalt to thickening agent is too high, then the product may be too hard, and may not release a sufficient amount of cosmetic material when the user wets the product.

Preferably, the weight ratio of isomalt to thickening agent is from about 10:1 to about 30:1, such as from about 10:1 to about 25:1, such as from about 10:1 to about 22.5:1. In some embodiments, the weight ratio of isomalt to thickening agent is from about 15:1 to about 25:1.

In some embodiments, the solid cosmetic composition comprises:
(i) isomalt in an amount of from about 50% to about 70% by weight of the solid cosmetic composition; and
(ii) a thickening agent in an amount of from about 1% to about 20% by weight of the solid cosmetic composition, wherein the thickening agent is selected from the group consisting of a hydrocolloid, a clay, and mixtures thereof.

In some embodiments, the solid cosmetic composition comprises:
(i) isomalt in an amount of from about 40% to about 90% by weight of the solid cosmetic composition; and
(ii) a thickening agent in an amount of from about 1% to about 30% by weight of the solid cosmetic composition, wherein the thickening agent is selected from the group consisting of xanthan gum, cationic guar gum, carrageenan, sodium alginate, and mixtures thereof.

In some embodiments, the solid cosmetic composition comprises:
(i) isomalt in an amount of from about 40% to about 90% by weight of the solid cosmetic composition; and
(ii) a thickening agent in an amount of from about 1% to about 30% by weight of the solid cosmetic composition, wherein the thickening agent is selected from the group consisting of xanthan gum, cationic guar gum, carrageenan, and mixtures thereof.

In some embodiments, the solid cosmetic composition comprises:
(i) isomalt in an amount of from about 50% to about 70% by weight of the solid cosmetic composition; and
(ii) a thickening agent selected from the group consisting of xanthan gum, cationic guar gum, carrageenan, and mixtures thereof in an amount of from about 1% to about 20% by weight of the solid cosmetic composition.

In some embodiments, the solid cosmetic composition comprises:
(i) isomalt in an amount of from about 50% to about 70% by weight of the solid cosmetic composition; and
(ii) a thickening agent selected from the group consisting of xanthan gum, cationic guar gum, carrageenan, sodium alginate, and mixtures thereof in an amount of from about 1% to about 20% by weight of the solid cosmetic composition.

In some embodiments, the solid cosmetic composition comprises:
(i) isomalt in an amount of from about 40% to about 90% by weight of the solid cosmetic composition; and
(ii) a thickening agent in an amount of from about 1% to about 30% by weight of the solid cosmetic composition,

wherein the weight ratio of isomalt to thickening agent is from about 10:1 to about 40:1, and wherein the thickening agent is selected from the group consisting of a hydrocolloid, a clay, and mixtures thereof.

In some embodiments, the solid cosmetic composition comprises:
(i) isomalt in an amount of from about 40% to about 90% by weight of the solid cosmetic composition; and
(ii) a thickening agent in an amount of from about 1% to about 30% by weight of the solid cosmetic composition, wherein the thickening agent is selected from the group consisting of xanthan gum, cationic guar gum, carrageenan, and mixtures thereof,

wherein the weight ratio of isomalt to the thickening agent is from about 10:1 to about 40:1.

In some embodiments, the solid cosmetic composition comprises:
(i) isomalt in an amount of from about 40% to about 90% by weight of the solid cosmetic composition;
(ii) a thickening agent in an amount of from about 1% to about 30% by weight of the solid cosmetic composition, wherein the thickening agent is selected from the group consisting of a hydrocolloid, a clay, and mixtures thereof; and
(iii) a humectant.

In some embodiments, the solid cosmetic composition comprises:
(i) isomalt in an amount of from about 40% to about 90% by weight of the solid cosmetic composition;
(ii) a thickening agent in an amount of from about 1% to about 30% by weight of the solid cosmetic composition, wherein the thickening agent is selected from the group consisting of xanthan gum, cationic guar gum, carrageenan, and mixtures thereof; and
(iii) a humectant selected from the group consisting of glycerine, propylene glycol, and mixtures thereof.

In some embodiments, the solid cosmetic composition comprises:
(i) isomalt in an amount of from about 50% to about 70% by weight of the solid cosmetic composition;
(ii) a thickening agent in an amount of from about 1% to about 20% by weight of the solid cosmetic composition, wherein the thickening agent is selected from the group consisting of a hydrocolloid, a clay, and mixtures thereof; and
(iii) a humectant in an amount of from about 5% to about 40% by weight of the solid cosmetic composition.

In some embodiments, the solid cosmetic composition comprises:
(i) isomalt in an amount of from about 50% to about 70% by weight of the solid cosmetic composition;
(ii) a thickening agent selected from the group consisting of xanthan gum, cationic guar gum, carrageenan, and mixtures thereof in an amount of from about 1% to about 20% by weight of the solid cosmetic composition; and
(iii) a humectant selected from the group consisting of glycerine, propylene glycol, and mixtures thereof in an amount of from about 5% to about 40% by weight of the solid cosmetic composition.

In some embodiments, the solid cosmetic composition comprises:
(i) isomalt in an amount of from about 40% to about 90% by weight of the solid cosmetic composition;
(ii) a thickening agent in an amount of from about 1% to about 30% by weight of the solid cosmetic composition, wherein the thickening agent is selected from the group consisting of a hydrocolloid, a clay, and mixtures thereof; and
(iii) an active agent.

In some embodiments, the solid cosmetic composition comprises:
(i) isomalt in an amount of from about 40% to about 90% by weight of the solid cosmetic composition;
(ii) a thickening agent in an amount of from about 1% to about 30% by weight of the solid cosmetic composition, wherein the thickening agent is selected from the group consisting of a hydrocolloid, a clay, and mixtures thereof;
(iii) a humectant; and
(iv) an active agent.

In some embodiments, the solid cosmetic composition comprises:
(i) isomalt in an amount of from about 40% to about 90% by weight of the solid cosmetic composition;
(ii) a thickening agent in an amount of from about 1% to about 30% by weight of the solid cosmetic composition, wherein the thickening agent is selected from the group consisting of a hydrocolloid, a clay, and mixtures thereof; and
(iii) a surfactant,

and optionally a humectant.

In some embodiments, the solid cosmetic composition comprises:
(i) isomalt in an amount of from about 40% to about 90% by weight of the solid cosmetic composition;
(ii) a thickening agent in an amount of from about 1% to about 30% by weight of the solid cosmetic composition, wherein the thickening agent is selected from the group consisting of a hydrocolloid, a clay, and mixtures thereof; and
(iii) an oral care agent,

and optionally a humectant.

In some embodiments, the solid cosmetic composition comprises:
(i) isomalt in an amount of from about 40% to about 90% by weight of the solid cosmetic composition;
(ii) a thickening agent in an amount of from about 1% to about 30% by weight of the solid cosmetic composition, wherein the thickening agent is selected from the group consisting of a hydrocolloid, a clay, and mixtures thereof; and
(iii) a hair gelling agent,

and optionally a humectant.

In some preferred embodiments, the composition comprises water in a total amount of no greater than about 15% by weight of the solid cosmetic composition, such as in a total amount of no greater than about 10% by weight of the solid cosmetic composition, such as in a total amount of no greater than about 5% by weight of the solid cosmetic composition, such as in a total amount of no greater than about 1% by weight of the solid cosmetic composition, such as in a total amount of no greater than 0.5% by weight of the solid cosmetic composition, such as in a total amount of no greater than 0.1% by weight of the solid cosmetic composition. As used herein the “total amount” of water includes any water that is provided by the surfactant product included in the composition. It was found by the present inventors that, by limiting the amount of water to no greater than 15% by weight in the solid product, the stability of the product and its shelf-life could be improved. Without wishing to be bound, this may be due in part to an improved resistance of the product to microbiological growth when low amounts of water are included, and in part to a decreased water content leading to a product that does not become tacky or sticky to touch after storage for an extended period of time.

Preferably, the solid cosmetic composition is suitable for use in washing the hair and/or body of the user. Preferably, the solid cosmetic composition is suitable for use in washing both the hair and body of the user. In some preferred embodiments, the solid cosmetic composition is a solid shower gel. In some preferred embodiments, the solid cosmetic composition is a solid shampoo.

Preferably, the solid cosmetic composition is a solid hair gel product. The solid cosmetic composition may thus be suitable for use in holding the user's hair in a predefined position, so as to enable styling and/or shaping of the hair.

Preferably, the solid cosmetic composition is a solid oral care product, which may be suitable for use in cleaning and/or whitening the user's teeth, or freshening the user's mouth.

Preferably, the solid cosmetic composition is a hair dye (or hair colouring) product. The solid cosmetic composition may thus be suitable for use in permanently, semi-permanently or temporarily colouring the hair of the user.

Preferably the solid cosmetic composition is suitable for use in colouring the skin of the user. Preferably, the solid cosmetic composition is a solid make-up product; for example, a solid foundation, solid eye shadow, solid lipstick, solid blush, solid bronzer, or the like.

The solid cosmetic composition may also be suitable for use as a skin moisturiser and/or hand/body lotion.

The above ranges provide preferred amounts of each of the components. Each of these ranges may be taken alone or combined with one or more other component ranges to provide a preferred aspect of the invention.

Further Broad Aspects

Also described herein, are the following broad aspects according to the disclosure.

There is described herein a solid cosmetic composition comprising:
(i) isomalt; and
(ii) a thickening agent.

There is described herein a solid cosmetic composition comprising:
(i) isomalt in an amount of from about 50% to about 70% by weight of the solid cosmetic composition; and
(ii) a thickening agent in an amount of from about 1% to about 20% by weight of the solid cosmetic composition.

There is described herein a solid cosmetic composition comprising:
(i) isomalt; and
(ii) a thickening agent selected from the group consisting of xanthan gum, cationic guar gum, carrageenan, and mixtures thereof.

There is described herein a solid cosmetic composition comprising:
(i) isomalt in an amount of from about 50% to about 70% by weight of the solid cosmetic composition; and
(ii) a thickening agent selected from the group consisting of xanthan gum, cationic guar gum, carrageenan, and mixtures thereof in an amount of from about 1% to about 20% by weight of the solid cosmetic composition.

There is described herein a solid cosmetic composition comprising:
(i) isomalt; and
(ii) a thickening agent,

wherein the weight ratio of isomalt to thickening agent is from about 10:1 to about 40:1.

There is described herein a solid cosmetic composition comprising:
(i) isomalt; and
(ii) a thickening agent selected from the group consisting of xanthan gum, cationic guar gum, carrageenan, and mixtures thereof,

wherein the weight ratio of isomalt to the thickening agent is from about 10:1 to about 40:1.

There is described herein a solid cosmetic composition comprising:
(i) isomalt;
(ii) a thickening agent; and
(iii) a humectant.

There is described herein a solid cosmetic composition comprising:
(i) isomalt;
(ii) a thickening agent selected from the group consisting of xanthan gum, cationic guar gum, carrageenan, and mixtures thereof; and
(iii) a humectant selected from the group consisting of glycerine, propylene glycol, and mixtures thereof.

There is described herein a solid cosmetic composition comprising:
(i) isomalt in an amount of from about 50% to about 70% by weight of the solid cosmetic composition;
(ii) a thickening agent in an amount of from about 1% to about 20% by weight of the solid cosmetic composition; and
(iii) a humectant in an amount of from about 5% to about 40% by weight of the solid cosmetic composition.

There is described herein a solid cosmetic composition comprising:
(i) isomalt in an amount of from about 50% to about 70% by weight of the solid cosmetic composition;
(ii) a thickening agent selected from the group consisting of xanthan gum, cationic guar gum, carrageenan, and mixtures thereof in an amount of from about 1% to about 20% by weight of the solid cosmetic composition; and
(iii) a humectant selected from the group consisting of glycerine, propylene glycol, and mixtures thereof in an amount of from about 5% to about 40% by weight of the solid cosmetic composition.

There is described herein a solid cosmetic composition comprising:
(i) isomalt;
(ii) a thickening agent; and
(iii) an active agent.

There is described herein a solid cosmetic composition comprising:
(i) isomalt;
(ii) a thickening agent;
(iii) a humectant; and
(iv) an active agent.

There is described herein a solid cosmetic composition comprising:
(i) isomalt;
(ii) a thickening agent; and
(iii) a surfactant,

and optionally a humectant.

There is described herein a solid cosmetic composition comprising:
(i) isomalt;
(ii) a thickening agent; and
(iii) an oral care agent,

and optionally a humectant.

There is described herein a solid cosmetic composition comprising:
(i) isomalt;
(ii) a thickening agent; and
(iii) a hair gelling agent,

and optionally a humectant.

Process

As described herein, according to a second aspect of the invention, there is provided a process for the production of a solid cosmetic composition as defined herein, the process comprising the steps of:
(a) combining:
   (i) isomalt, and
   (ii) a thickening agent; and
(b) solidifying the resultant combination.

In some embodiments, the step (a) comprises combining the isomalt and thickening agent at a temperature of from about 90° C. to about 200° C., such as from about 100° C. to about 170° C., such as from about 100° C. to about 150° C., such as from about 100° C. to about 140° C.

In some preferred embodiments, the isomalt may first be heated to a temperature of from about 145° C. to about 190° C. in order to melt the isomalt. Preferably, the isomalt is first heated to a temperature of from about 150° C. to about 170° C. The isomalt may then be cooled slightly, prior to combining with the thickening agent. For example, the isomalt may initially be heated to a temperature of from about 145° C. to about 190° C. (preferably from about 150° C. to about 170° C.), and then combined with the thickening agent at a temperature of from about 90° C. to about 140° C., such as from about 90° C. to about 120° C., such as approximately 100° C.

In some embodiments, the mixture of step (a) is poured, pulled, spun or blown. Such techniques will be apparent to one skilled in the art of preparing sugar or sugar replacement products. In some embodiments, the mixture of step (a) is extruded or 3D printed prior to solidifying.

The shape of the solid compositions of the present invention is not limited. It may be that the solid compositions are provided with a shape that would be aesthetically pleasing and/or which aids in the use of the product. For example, it may be that the solid product is produced in such a manner so that it solidifies in a shape which is ergonomically acceptable to the user. Therefore, in some embodiments of the process of the present invention, the mixture of step (a) is poured and/or pressed into a mould, allowed to solidify, and then turned out to produce the solid product.

In some embodiments, the process comprises the steps of
(a) combining:
   (i) isomalt in an amount of from about 40% to about 90% by weight of the combination, and
   (ii) a thickening agent in an amount of from about 1% to about 30% by weight of the combination; and
(b) solidifying the resultant combination.

As described herein, according to a third aspect of the invention, there is also provided a solid cosmetic composition obtained or obtainable from the process as described herein.

Method

As described herein, according to a fourth aspect of the invention, there is provided a cosmetic method comprising contacting the skin, hair or teeth of a user with the solid cosmetic composition as defined herein. As will be appreciated by the skilled person, in view of the solid properties of the present invention, the user may contact the skin, hair or teeth directly with the solid cosmetic composition. There may be no need to dispense the product from within some external packaging prior to application to the skin, as is the case for conventional liquid shower gel, shampoo, hair gel and oral care products.

Preferably, the solid cosmetic composition may be contacted with the skin, hair or teeth of a user via a method comprising:
(i) contacting the solid cosmetic composition with water,
(ii) applying some pressure to the solid cosmetic composition to form a semi-solid or liquid substance (e.g. a gel or paste) on the surface of the composition, and
(iii) applying the semi-solid or liquid substance to the skin, hair or teeth of a user.

Preferably, pressure may be applied by the user rubbing the product with their hands or fingers. Alternatively, pressure may be applied with a toothbrush, cosmetic brush, colour applicator or other appliance which is subsequently used to apply the product to the skin, hair or teeth of the user.

Preferably, only the material at the surface of the composition is transformed into a semi-solid or liquid substance. After use, the product may be left to dry; i.e. the surface of the product may dry such that the semi-solid or liquid substance re-solidifies. The solid cosmetic composition may then be reused by the user until all of the cosmetic material has been used by the user.

Use

Also described herein is the use of isomalt for preparing a solid cosmetic composition that releases a cosmetically active agent upon contact with water.

### EXAMPLES

The invention will now be described with reference to the following non-limiting examples.

### Example 1

A product having the following composition was prepared:

| | Formula | Batch Size (g): |
|---|---|---|
| Component | (% by weight) | 100.00 |
| Isomait | 64 | 64.00 |
| Glycerine | 25 | 25.00 |
| Xanthan gum | 3 | 3.00 |
| PVP | 3 | 3.00 |
| Cetrimonium Chloride | 3 | 3.00 |
| Fragrance | 2 | 2.00 |
| TOTAL | 100 | 100.00 |

The product was prepared using the following method:
1. The isomalt was heated to a temperature of 165° C.
2. The isomalt was removed from the heat, and cooled to 140° C.
3. The glycerine was then added, and the mixture stirred gently. PVP and cetrimonium chloride was then added, and the mixture stirred again.
4. When the mixture had cooled to 100° C., the xanthan gum and fragrance were added, and the resulting mixture stirred gently.
5. The resultant combination was then poured into a mould, and left to set.

It was found that a solid product was obtained that was not tacky to the touch when no water had been applied (externally, by a user) to the product. Upon contacting a small amount of water to the product and the user contacting the product with their hand (e.g. by the user wetting their hands, and then touching or rubbing the product with their wet hand), the user was able to release the cosmetically active ingredients from within the solid composition. The PVP and cetrimonium chloride was thus released with a gel-type texture on the surface of the solid product, which gel-type active the user could then apply to their hair as they would with a conventional hair gel. The composition was found to provide acceptable gelling properties upon activation of the product by application of only a small amount of water.

### Examples 1 to 12 and Comparative Example 1 to 6

The following products were prepared having the following compositions. Each product was prepared using the method described above for Example 1. It is noted that Examples 1 to 12 fall within the scope of the claimed invention, whilst Comparative Examples 1 to 6 fall outside the scope of the claimed invention.

| | Formula (% by Weight) | | | | | |
|---|---|---|---|---|---|---|
| | Example | Example | Example | Example | Example | Example |
| Component | 1 | 2 | 3 | 4 | 5 | 6 |
| Isomalt | 64 | 76 | 89 | 50 | 64 | 64 |
| Glycerine | 25 | 13 | — | 39 | — | — |
| Propylene | — | — | — | — | 25 | — |
| Glycol | | | | | | |
| Sodium | — | — | — | — | — | 25 |
| laureth | | | | | | |
| sulfate | | | | | | |
| PEG-7 | — | — | — | — | — | — |
| glyceryl | | | | | | |
| cocoate | | | | | | |
| Xanthan | 3 | 3 | 3 | 3 | 3 | 3 |
| Gum | | | | | | |
| Bentonite | — | — | — | — | — | — |
| PVP | 3 | 3 | 3 | 3 | 3 | 3 |
| Cetrimonium | ³ | 3 | 3 | 3 | 3 | 3 |
| chloride | | | | | | |
| Fragrance | 2 | 2 | 2 | 2 | 2 | 2 |
| TOTAL | 100 | 100 | 100 | 100 | 100 | 100 |

| | Formula (% by Weight) | | | | | |
|---|---|---|---|---|---|---|
| | Example | Example | Example | Example | Example | Example |
| Component | 7 | 8 | 9 | 10 | 11 | 12 |
| Isomalt | 64 | 64 | 70 | 95 | 95 | 70 |
| Glycerine | — | 13 | 25 | — | — | 25 |
| Propylene | — | — | — | — | — | — |
| Glycol | | | | | | |
| Sodium | — | — | — | — | — | — |
| laureth | | | | | | |
| sulfate | | | | | | |
| PEG-7 | 25 | — | — | — | — | — |
| glyceryl | | | | | | |
| cocoate | | | | | | |
| Xanthan | 3 | 15 | 3 | 3 | — | — |
| Gum | | | | | | |
| Bentonite | — | — | — | — | 3 | 3 |
| PVP | 3 | 3 | — | — | — | — |
| Cetrimonium | 3 | 3 | — | — | — | — |
| chloride | | | | | | |
| Fragrance | 2 | 2 | 2 | 2 | 2 | 2 |
| TOTAL | 100 | 100 | 100 | 100 | 100 | 100 |

| | Formula (% by Weight) | | | | | |
|---|---|---|---|---|---|---|
| | Comparative | Comparative | Comparative | Comparative | Comparative | Comparative |
| Component | Example 1 | Example 2 | Example 3 | Example 4 | Example 5 | Example 6 |
| Isomalt | — | — | — | — | 64 | 64 |
| Sorbitol | 64 | — | — | — | — | — |
| Xylitol | — | 64 | — | — | — | — |
| Mannitol | — | — | 64 | — | — | — |
| Erythritol | — | — | — | 64 | — | — |
| Glycerine | 25 | 25 | 25 | 25 | 28 | 19 |
| Xanthan | 3 | 3 | 3 | 3 | — | — |
| Gum | | | | | | |
| PVP | 3 | 3 | 3 | 3 | 3 | — |
| Cetrimonium | 3 | 3 | 3 | 3 | 3 | — |
| chloride | | | | | | |
| Calcium | — | — | — | — | — | 15 |
| carbonate | | | | | | |
| Fragrance | 2 | 2 | 2 | 2 | 2 | 2 |
| TOTAL | 100 | 100 | 100 | 100 | 100 | 100 |

| Example | Observations |
|---|---|
| 1 | The product was solid, and was not tacky on the outside prior to wetting. Upon wetting of the product with water and |
| | subsequent rubbing of the product in the user's hand, an acceptable amount of gel was released from the product, which |
| | could be rubbed onto the user's hair to provide a hair gelling effect. |
| 2 | The product was solid, and was not tacky on the outside prior to wetting. Upon wetting of the product with water and |
| | subsequent rubbing of the product in the user's hand, an acceptable amount of gel was released from the product, which |
| | could be rubbed onto the user's hair to provide a hair gelling effect. |
| 3 | The product was solid and very hard. There was no tackiness at all prior to wetting. Upon wetting of the product with water |
| | and subsequent rubbing of the product in the user's hands, a small amount of gel was released from the product. |
| 4 | The product was solid. Prior to wetting, the product had a degree of tackiness to the touch and was not completely dry. |
| | Upon wetting of the product with water and subsequent rubbing of the product in the user's hand, an acceptable amount of |
| | gel was released from the product, which could be rubbed onto the user's hair to provide a hair gelling effect. |
| 5 | The product was solid, and was not tacky on the outside prior to wetting. Upon wetting of the product with water and |
| | subsequent rubbing of the product in the user's hand, an acceptable amount of gel was released from the product, which |
| | could be rubbed onto the user's hair to provide a hair gelling effect. Less gel was released than for Example 1, but the |
| | amount released was still acceptable to provide the hair gelling effect. |
| 6 | The product was solid. Prior to wetting, the product was slightly tacky to the touch and had not fully dried out. Upon wetting |
| | of the product with water and subsequent rubbing of the product in the user's hand, a relatively large amount of gel was |
| | released from the product, which could be rubbed onto the user's hair to provide a hair gelling effect. |
| 7 | The product was solid, and was not tacky on the outside prior to wetting. Upon wetting of the product with water and |
| | subsequent rubbing of the product in the users hand, an acceptable amount of gel was released from the product, which |
| | could be rubbed onto the user's hair to provide a hair gelling effect. |
| 8 | The product was solid, and was not tacky on the outside prior to wetting. Upon wetting of the product with water and |
| | subsequent rubbing of the product in the user's hand, an acceptable amount of gel was released from the product, which |
| | could be rubbed onto the user's hair to provide a hair gelling effect. It was found in particular that, when this product was |
| | left in a beaker of water for a short period of time, a very large amount of gel was released from the product to give a very |
| | good hair gelling effect. |
| 9 | The product was solid, and was not tacky on the outside prior to wetting. Upon wetting of the product with water and |
| | subsequent rubbing of the product in the user's hand, an acceptable amount of gel was released from the product. |
| 10 | The product was solid and harder than other a number of the other Examples, and was slightly tacky on the outside prior to |
| | wetting. The product almost appeared translucent. Upon wetting of the product with water and subsequent rubbing of the |
| | product in the user's hand, a reasonable amount of gel was released from the product. |
| 11 | The product was solid. Prior to wetting, the product had a degree of tackiness to the touch and was not completely dry. |
| | Upon wetting of the product with water and subsequent rubbing of the product in the user's hand, a slight amount of gel |
| | was released from the product. The amount of gel released was less than Example 10, but was still considered to be |
| | acceptable. |
| 12 | The product was solid, and was not tacky on the outside prior to wetting. Upon wetting of the product with water and |
| | subsequent rubbing of the product in the user's hand, a slight amount of gel was released from the product. |
| Comparative | The product was solid, but very crumbly. It was not possible for the user to rub the solid in their hands without the product |
| Example 1 | crumbling. |
| Comparative | The product was a liquid, and could not be rubbed in the user's hands. |
| Example 2 | |
| Comparative | The product was solid, but was formed in lumps such that it was not possible to pour the product into a mould to provide an |
| Example 3 | acceptable finished product that could be held in the hand of the user. The product was not tacky, and released a small |
| | amount of gel upon wetting. |
| Comparative | The product was semi-solid (paste-like and “mushy”), but was very tacky on the outside - humectant was observed to be |
| Example 4 | leaking out of the product. |
| Comparative | The product was solid. However, upon wetting of the product with water and subsequent rubbing of the product in the |
| Example 5 | user's hand, very little gel was released from the product. The amount of not enough to be able to apply to the user's hair in |
| | order to provide the desired gelling effect. |
| Comparative | The product was solid. However, upon wetting of the product with water and subsequent rubbing of the product in the |
| Example 6 | user's hand, very little gel was released from the product. The amount of calcium carbonate as the active agent released |
| | from the product was therefore considered to be inadequate. |

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the claims.

1. A solid cosmetic composition comprising
(i) isomalt in an amount of from about 40% to about 90% by weight of the solid cosmetic composition, and
(ii) a thickening agent in an amount of from about 1% to about 30% by weight of the solid cosmetic composition, and wherein the thickening agent is selected from the group consisting of a hydrocolloid, a clay, and mixtures thereof.

2. The solid cosmetic composition according to claim 1, wherein the isomalt is present in an amount of from about 45% to about 80% by weight of the solid cosmetic composition.

3. The solid cosmetic composition according to claim 1, wherein the isomalt is present in an amount of from about 50% to about 70% by weight of the solid cosmetic composition.

4. The solid cosmetic composition according to claim 1, wherein the thickening agent is present in an amount of from about 1% to about 20% by weight of the solid cosmetic composition.

5. The solid cosmetic composition according to claim 1, wherein the thickening agent is selected from the group consisting of xanthan gum, guar gum, cationic guar gum, agar, pectin, a salt of alginic acid, carrageenan, Arabic gum, acacia gum, and mixtures thereof.

6. The solid cosmetic composition according claim 1, wherein the thickening agent is xanthan gum.

7. The solid cosmetic composition according to claim 1, wherein the weight ratio of isomalt to thickening agent is from about 10:1 to about 40:1.

8. The solid cosmetic composition according to claim 1, further comprising a humectant.

9. The solid cosmetic composition according to claim 8, wherein the humectant is present in an amount of from about 5% to about 40% by weight of the solid cosmetic composition.

10. The solid cosmetic composition according to claim 8, wherein the humectant is selected from the group consisting of glycerine, propylene glycol, 1,3-propanediol, honey, sorbitol, and mixtures thereof.

11. The solid cosmetic composition according to claim 8, wherein the weight ratio of isomalt to humectant is from about 1:1 to about 5:1.

12. The solid cosmetic composition according to claim 1, further comprising a surfactant.

13. The solid cosmetic composition according to claim 12, wherein the surfactant is selected from the group consisting of sodium lauryl sulfate, sodium laureth sulfate, sodium cocosulfate, disodium laureth sulfosuccinate, disodium lauryl sulfosuccinate, sodium lauryl sulfoacetate, lauryl betaine, cocamidopropyl betaine, sodium cocoamphoacetate, disodium cocoamphodiacetate, sodium lauroyl sarcosine, cetrimonium chloride and mixtures thereof.

14. The solid cosmetic composition according to claim 12, wherein the surfactant is present in an amount of from about 1% to about 30% by weight of the solid cosmetic composition.

15. The solid cosmetic composition according to claim 1, wherein the composition is a solid hair gel.

16. The solid cosmetic composition according to claim 1, further comprising polyvinylpyrrolidone, polyvinylpyrrolidone and dimethylaminoethylmethylacrylate copolymer, carbomer, vinylpyrrolidone and vinyl acetate copolymer, vinylpyrrolidone and dimethylaminopropylamine Acrylates copolymer, polyacrylate-2-crosspolymer, sodium polyitaconate, vinylpyrrolidone/methacrylamide/vinyl imidazole copolymer, or mixtures thereof.

17. The solid cosmetic composition according to claim 1, wherein the composition is a solid oral care composition.

18. The solid cosmetic composition according to claim 1, further comprising an oral care agent selected from the group consisting of calcium carbonate, aluminium hydroxide, calcium hydrogen phosphate, silica, zeolite, sodium fluoride, stannous fluoride, olaflur, sodium monofluorophosphate, xylitol, sodium bicarbonate, and mixtures thereof.

19. The solid cosmetic composition according to claim 1, wherein the solid cosmetic composition comprises water in an amount of no greater than about 5% by weight of the solid cosmetic composition.

20. The solid cosmetic composition according to claim 1, further comprising one or more cosmetically acceptable additives selected from a binder, filler, exfoliating material, essential oil, vitamin, perfume, fragrance, colouring, decorative item, effervescent component, and mixtures thereof.

21. A process for the production of a solid cosmetic composition as defined in claim 1, the process comprising the steps of:
(a) combining:
(i) isomalt, and
(ii) a thickening agent; and
(b) solidifying the resultant combination.

22. The process according to claim 21, wherein the step (a) comprises combining isomalt and thickening agent at a temperature of from about 90° C. to about 200° C.

23. A solid cosmetic composition obtained or obtainable by the process of claim 21.

24. A cosmetic method comprising contacting the skin, hair or teeth of a user with the solid cosmetic composition as defined in claim 1.
